(19) United States
(12) Patent Application Publication
(10) Pub. No.: US 2005/0100902 A1
(43) Pub. Date: May 12, 2005

(54) 5-cyano-1h-indole derivatives as antagonist of the inerleukine-8 receptors

(76) Inventors: Martine Barth, Asnieres les Dijon (FR); Pierre Dodey, Fontaine Les Dijon (FR); Jean-Luc Paquet, Brognon (FR)

Correspondence Address: MERCHANT & GOULD PC, P.O. BOX 2903, MINNEAPOLIS, MN, 55402-0903, US

(21) Appl. No.: 10/477,650

(22) PCT Filed: May 16, 2002
(86) PCT No.: PCT/FR02/01647

(30) Foreign Application Priority Data
May 17, 2001 (FR) 01/06506

(51) Int. Cl. C12Q 1/68

(52) U.S. Cl. 435/6

(57) ABSTRACT

The present invention relates to derivatives of 5-cyano-1H-indole of formula (I): 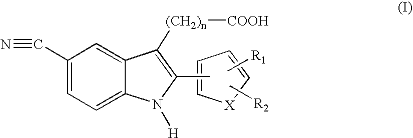
in which R₁, R₂, X and n are as defined in claim 1, as well as to their pharmaceutically acceptable salts, solvates and hydrates. Pharmaceutical compositions containing them, as well as their use for the preparation of medicaments intended for the preventative or curative treatment of illnesses which are dependent upon the activation of the interleukin CXCR2 receptor and of the chemokines of the same family, are also subjects of the invention.

The present invention relates to novel derivatives of 5-cyano-1H-indole, to pharmaceutical compositions containing them, as well as to their use for the preparation of medicaments which are intended for treating illnesses which are dependent upon interleukin-8 receptors.

IL-8 (Interleukin-8) is a protein of 72 amino acids which belongs to the superfamily of proteins which are capable of attracting leukocytes, which are also called C—X—C or C—C cytokines, intercrine cytokines or, more recently, chemokines (Oppenheim et al., Annu. Rev Immunol., 1991. 9, 617-648). Various names have been given to interleukin-8, such as NAP-1 (“neutrophil activating peptide-1”), NAF (“neutrophil activating factor”) and “T-cell lymphocyte chemotactic factor”. Numerous members of the family of chemokines have been described as being involved in inflammatory processes and in the migration of leukocytes. The chemokine family is composed of two distinct sub-families: alpha- and beta-chemokines. Alpha-chemokines, such as IL-8, NAP-2 (“Neutrophil activating peptide-2”), MGSA/Gro, or Gro-alpha (“melanoma growth stimulatory activity”), and ENA-78 (“Epithelial cell derived neutrophil activating protein 78”), have all the effects upon the attraction and the activation of leukocytes and more particularly of neutrophils. This sub-family also includes PF-4 (“Platelet Factor-4”), beta-thromboglobulin and CTAPIII (“connective tissue activating protein III”), which themselves do not have any effect upon neutrophils.

IL-8 was originally identified by its capacities to attract and to activate polymorphonuclear leukocytes (neutrophils). More recently, it has been demonstrated that the expression of IL-8 was induced rapidly in various tissues or cells such as macrophages, fibroblasts, endothelial and epithelial cells and even neutrophils, in response to pro-inflammatory cytokines such as alpha or beta IL-1 or TNF alpha (“Tumor necrosis factor”) or to other pro-inflammatory agents such as LPS (“Lipopolysaccharide”) (Van Damme J., Interleukin-8 and related chemotactic cytokines; 1994; The Cytokines Handbook, 2^{nd} Ed. A. W. Thomson editor, Academic Press, London, pp: 185-208). Further, certain data from the literature have demonstrated high systemic levels of IL-8 in certain inflammatory pathologies which involve neutrophils, suggesting that IL-8 and other chemokines of the same family can be fundamental mediators of the activation of neutrophils (Van Damme, Interleukin-8 and related chemotactic cytokines; 1994; The Cytokines Handbook, 3^{eme} Ed. A. W. Thomson editor, Academic Press, London, pp: 271-311).

Gro-alpha, Gro-beta, Gro-gamma and NAP-2 belong to the family of chemokines and, as IL-8, these proteins have also been designated by various terms. Thus, Gro-alpha, beta and gamma have been called, respectively, MGSA (“Melanoma Growth Stimulatory Activity”) a, b and g (Richmond and Thomas, J. Cell Physiol., 1986. 129, 375-384; Cheng et al., J. Immunol, 1992. 148. 451-456). All these chemokines belong to the group of alpha-chemokines which possess an ELR (Aspartate-Leucine-Arginate) moiety further up from the CXC moiety which is characteristic of this subgroup. These chemokines all bind to the type 2 receptor or CXCR2.

Two IL-8 receptors belonging to the family of G-protein coupled transmembrane seven-domain receptors have been characterised and cloned type A IL-8 receptor (IL-8RA), or CXCR1, which binds with high affinity the IL-8 and GCP-2 (<<granulocyte chemoattractant protein 2>>), and type B IL-8 receptor (IL-8RB), or CXCR2, which has, as specific ligands, IL-8, GCP-2, Gro-alpha, Gro-beta, Gro-gamma, and NAP-2 (Ponath, Exp. Opin. Invest. Drugs, 1998. 7, 1-18). These two receptors possess an amino acid sequence homology of 77%. Numerous publications have shown abnormally high levels of IL-8 in rheumatoid arthritis, septic shock, asthma, mucoviscidosis, myocardial infarction, and psoriasis (Baggiolini et al., FEBS Lett, 1992, 307, 97-101; Mille and Krangel., Crit. Rev. Immunol, 1992, 12, 17-46; Oppenheim et al., Annu. Rev. Immunol, 1991, 9, 617-648; Seitz et al., J. Clin. Invest, 1991, 87, 463-469; Miller et al, Am. Rev. Resp, Dis., 1992, 146, 427-432; Donnelly et al., Lancet, 1993, 341, 643-647). IL-8 seems to be involved in phenomena of ischaemia-reperfusion of the lung (Sekido et al, Nature, 1993, 365, 654-657). An antibody directed against IL-8 having the capacity to block the in vitro migration of rabbit neutrophils induced by IL-8, prevents tissular damage resulting from a process of pulmonary ischaemia/reperfusion in the rabbit. IL-8 seems to play a major role in the changes which are due to a hypoxia/reperfusion of the myocardium (Kukielka et al, J. Clin. Invest, 1995, 95, 89-103).

Another study has demonstrated beneficial effects of an IL-8 neutralising antibody in a endotoxin-induced pleurisy model in the rabbit (Broadus et al, J. Immunol., 1994, 152, 2960-2967). The involvement of IL-8 in inflammations of the lung as well as its deleterious role have been demonstrated with the aid of IL-8 neutralising antibodies in a model of pulmonary attack induced by an instillation of acid in the lungs of the rabbit (Folkesson et al., J. Clin. Invest, 1995, 96, 107-116) and in an endotoxin-induced acute respiratory distress syndrome model (Yokoi et al., Lab. Invest, 1997, 76, 375-384). Other reports have demonstrated similar beneficial effects with IL-8 neutralising antibodies in animal models of dermatosis, arthritis and of glomerulonephritis (Akahoshi et al., Lymphokine and Cytokine Res., 1994, 13, 113-116; Nishimura et al., J. Leukoc. Biol., 1997, 62, 444-449; Wada et al, J. Exp. Med., 1994, 180, 1135-1140). Furthermore, interleukin-8 receptor-deficient mice were generated by removal of the gene encoding the murine IL-8 receptor which is homologous to the human type 2 receptor (CXCR2) (Cacalano et al. Science, 1994, 265, 682-684). Although these mice are healthy, the characteristics of their neutrophils have been modified. Their capacity of migration into the peritoneum is in fact decreased in response to an intra-peritoneal injection of thioglycolate.

All these results show that the chemokines of the IL-8 family are important mediators of the migration and of the activation of neutrophils and of other cell types such as endothelial cells under certain inflammatory conditions. Furthermore, the chemokines of the IL-8 family have been described as playing an important role in tumour growth, the formation of metastases and tumoral angiogenesis in many types of cancer (Hebert and Baker, Cancer Invest, 1993, 11, 743-750; Richards et al., Am. J. Surg., 1997, 174, 507-512).

Certain compounds which are capable of binding to IL-8 receptors are described in the prior art: WO 96/18393, for example, discloses derivatives of 1-benzyl-2-indolecarboxylic acid, which are capable of binding to certain IL-8 receptors with an inhibiting effect. More recently, according to WO 99/06354, urea derivative or thiourea derivative compounds have also been presented as IL-8 receptor antagonists.

Furthermore, the patent application published under the number WO 00/51984 discloses certain indole derivatives of formula (A) 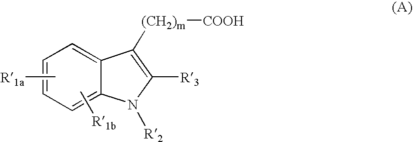
which are useful as intermediates in the synthesis of tachykinin antagonists.

However, it is to be noted that no compound of formula (A) is described in which R′₁ₐ or R′_{1b} represents a cyano group in position 5.

The invention provides novel non-peptidic compounds, which are derivatives of 5-cyano-1H-indole and which have the property of binding to the IL-8 CXCR2 receptor and other chemokines of the same family such as NAP-2, Gro-alpha or ENA-78, in behaving as antagonists.

A subject of the present invention is thus novel derivatives of 5-cyano-1H-indole of formula (I): 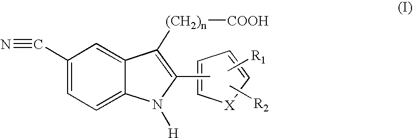
in which
   X represents a double bond —C═C— or a sulphur atom,
   R₁ and R₂ represent, each one independently of the other, a hydrogen atom, a halogen atom or a (C₁-C₃)alkyl, (C₁-C₃)alkoxy, trifluoromethyl, trifluoromethoxy, cyano or nitro group,
   n is equal to 2 or 3,
as well as their pharmaceutically acceptable salts, solvates and hydrates.

<<Alkyl >> is understood as meaning a linear or branched, saturated, monovalent hydrocarbon radical.

<<(C₁-C₃)alkyl >> is understood as meaning an alkyl radical comprising 1 to 3 carbon atoms.

<<Halogen atom >> is understood as meaning a fluorine, iodine, chlorine or bromine atom, fluorine and chlorine atoms being preferred.

The compounds which are currently preferred amongst the compounds of the invention are compounds of formula (Ia) 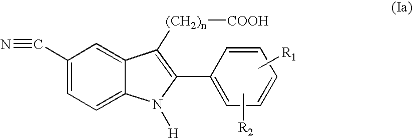
in which R₁, R₂ and n are as defined for (I), as well as their pharmaceutically acceptable salts, solvates and hydrates.

The compounds which are currently particularly preferred are compounds of formula (Ib): 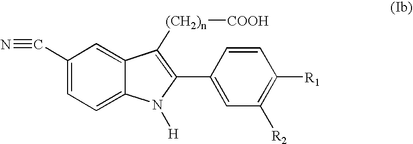
in which R₁, R₂ and n are as defined for (I), as well as their pharmaceutically acceptable salts, solvates and hydrates.

The preferred compounds of formula (I), (Ia) and (Ib) are those for which R₁ and R₂ represent, each independently, a hydrogen, chlorine or fluorine atom or a (C₁-C₂)alkyl, methoxy, trifluoromethyl, trifluoromethoxy, cyano or nitro group, n being equal to 2 or 3, as well as their pharmaceutically acceptable salts, solvates and hydrates.

Amongst these latter preferred compounds, the compounds which are more preferred are those for which R₁ and R₂ represent, each independently, a hydrogen, chlorine or fluorine atom or a methyl group, n being equal to 2 or 3, as well as their pharmaceutically acceptable salts, solvates and hydrates.

More particularly preferred compounds of formula (I), (Ia) and (Ib) are those for which at least one of the following conditions is fulfilled
n is equal to 3, and
R₁ represents a chlorine or fluorine atom, as well as their pharmaceutically acceptable salts, solvates and hydrates.

The compounds of formula (I), (Ia) and (Ib) can be salified with a pharmaceutically acceptable inorganic or organic base, according to techniques which are well known to the person skilled in the art. The term <<inorganic base >> is understood as meaning alkali metal hydroxides, such as soda, potash, lithia, or alkaline earth metal hydroxides, such as lime. The term <<organic base >> is understood as meaning primary, secondary or tertiary amines, amino-alcohols, certain non-toxic nitrogen-containing heterocycles, as well as basic amino acids. Amongst the salts, sodium salts or potassium salts, and salts of lysine, arginine or 2-amino-2-methyl-1,3-propanediol, are preferred.

The compounds of formula (I) according to the invention are, for example, prepared according to Scheme 1 below, in which R₁, R₂, X and n are as defined for (I), R₃ represents a (C₁-C₄)alkyl group, and Y represents a bromine or iodine atom.

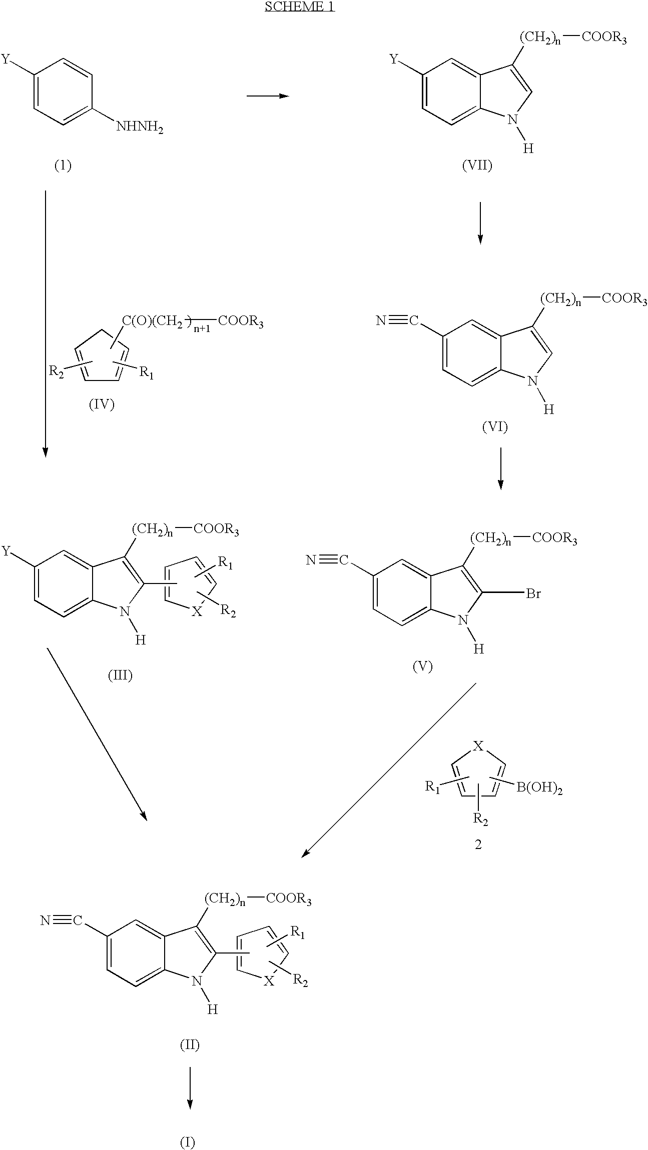

The compounds of formula (I) can be prepared by hydrolysis of the corresponding esters of formula: 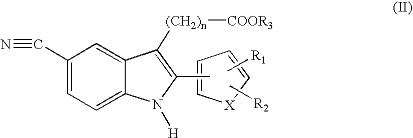
in which R₁, R₂, X and n are as defined for (I) and R₃ represents a (C₁-C₄)alkyl group, particularly a methyl or ethyl group.

Compounds (II) are novel intermediates and make up an integral part of the invention.

The hydrolysis of the compounds (II) into acid (I) is carried out according to techniques which are well known to the person skilled in the art, e.g. by the action of a hydro-alcoholic solution of sodium hydroxide.

The compounds of formula (II) can be prepared according to the following method
a) either by conversion of the compound of formula (III): 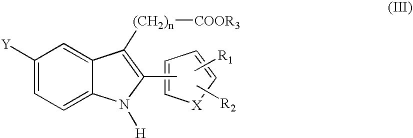
in which R₁, R₂, X and n are as defined for (I), R₃ represents a (C₁-C₄)alkyl group and Y represents a bromine or iodine atom, by the action of a cyanide,
b) or by a Suzuki coupling between the bromide derivative of formula (V): 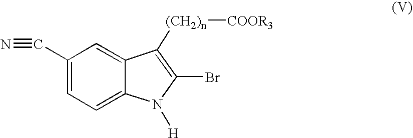
in which n is as defined for (I) and R₃ represents a (C₁-C₄)alkyl group, and the boronic acid of formula (2): 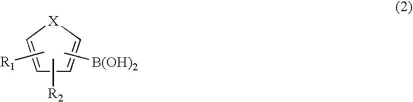
in which R₁, R₂ and X are as defined for (I), in the presence of a palladium catalyst such as tetrakis(triphenylphosphine)palladium.

In the step described in a), it will be possible for example to allow cuprous cyanide to react in the presence of N-methyl-2-pyrrolidone. It will also be possible to use potassium cyanide in the presence of a palladium catalyst. In this case, the reaction will be carried out for example in the presence of tetrakis(triphenylphosphine)palladium and copper iodide in a solvent such as tetrahydrofuran.

The step described in b) is preferably carried out in the presence of lithium chloride and sodium carbonate.

The compounds of formula (III) are, for example, obtained by a Fischer reaction between the compound of formula (IV): 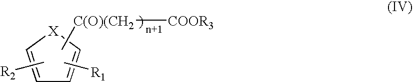
in which n, R₁, R₂ and X are as defined for (I), and R₃ represents a (C₁-C₄)alkyl group, with a phenylhydrazine of formula (1): 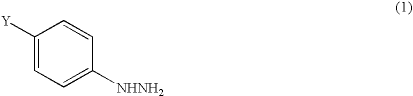
in which Y represents a bromine or iodine atom.

This Fischer reaction is carried out for example in the presence of zinc dichloride in acetic acid, at a temperature of between 20 and 80° C.

The compounds of formula (1) are commercial or are obtained according to techniques which are well known to the person skilled in the art.

The compounds (IV) can be obtained for example:
a) either by esterification, according to a reaction which is well known to the person skilled in the art, by the action of alcohol R₃OH in which R₃ represents a (C₁-C₄)alkyl group, on the acid of formula 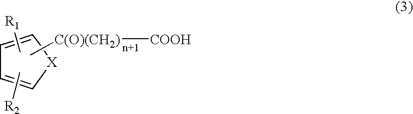
in which R₁, R₂, X and n are as defined for (I), it being possible for said acid (3) to be obtained by a Friedel Crafts-type reaction between a cyclic diacid anhydride of formula: 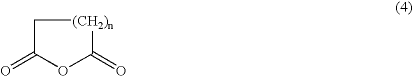
in which n is as defined for (I), with a compound of formula: 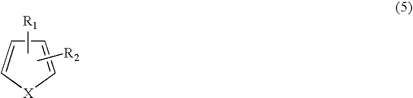
in which R₁, R₂ and X are as defined for (I), in the presence of a Lewis acid; it will be possible for example to carry out the reaction in the presence of aluminium trichloride in a solvent such as dichloromethane,
b) or directly by a Friedel Crafts-type reaction between an acid chloride of formula
   Cl—C(O)(CH₂)ₙ₊₁—COOR₃ (6)
in which n is as defined for (I) and R₃ represents a (C₁-C₄)alkyl group, with the compound of formula (5), in the presence of a Lewis acid, such as aluminium trichloride for example.

The compounds of formula (V) can be prepared by bromination, e.g. by the action of N-bromosuccinimide, of the compound of formula (VI): 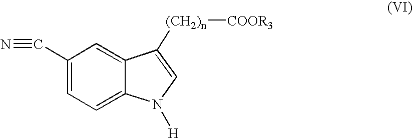
in which n is as defined for (I) and R₃ represents a (C₁-C₄)alkyl group.

The compounds of formula (VI) are for example prepared from the halogenated derivative of formula: 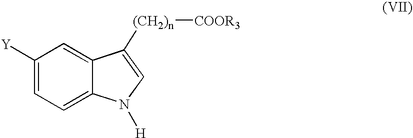
in which Y represents a bromine or iodine atom, preferably an iodine atom, by the action of a cyanide such as potassium cyanide, in the presence of a palladium catalyst. The reaction will be carried out for example in the presence of tetrakis(triphenylphosphine)palladium and copper iodide in a solvent such as tetrahydrofuran.

The compounds (VII) can be prepared according to a method which is analogous to the one used for the preparation of compounds (III), namely a Fischer reaction between a hydrazine (1) and an aldehyde of formula HC(O)(CH₂)ₙ₊₁—COOR₃ (7) in which n is as defined for (I) and R₃ represents a (C₁-C₄)alkyl group.

The boronic acids (2) used are commercial or known compounds.

The compounds of formula (I) according to the invention have been the subject of biological studies. Their inhibitory effect on the chemokines IL-8 and Gro-alpha was determined by the following in vitro tests:

A) IL-8 Receptor Binding Test

125-iodine labelled human IL-8 ([¹²⁵I]-IL-8) (NEN, Les Ulis, France) possesses a specific activity neighbouring 2,200 Ci/mmol. The recombinant human CXCR2 receptor was expressed in HEK 293 (ATCC, CRL-1573), K-562 (ATCC, CCL-243) or THP-1 (ATCC, TIB-202) cells. The HEK 293 cells are kept in culture in DMEM medium (<<Dulbecco modified eagle's medium>>) (GIBCO) containing 4.5 g/l of glucose, 10% of foetal calf serum, 1% of Glutamax, 1% of non-essential amino acids, 1 mM of sodium pyruvate, 100 IU/ml of penicillin and 100 μg/ml of streptomycin. The K-562 and THP-1 cells are kept in culture in RPMI1640 medium (GIBCO) containing 10% of foetal calf serum, 1% of non-essential amino acids, 1 mM of sodium pyruvate, 100 IU/ml of penicillin and 100 μg/ml of streptomycin. The cells are used when the cultures attain 80% of confluence.

The membranes are prepared according to the previously described protocol (Bastian et al Br. J. Pharmacol. 1997, 122, 393-399) except the homogenisation buffer which was replaced by a saline solution buffered at pH 8.0 containing 20 mM of Tris (tris(hydroxymethyl)aminomethane), 1.2 mM of MgSO₄ (magnesium sulphate), 0.1 mM of EDTA (ethylenediaminetetraacetic acid) and 25 mM of NaCl (sodium chloride). The competition experiments were carried out in plates of 96 wells of 1 ml, at ambient temperature, under a final volume of 0.25 ml. The membranes diluted in a solution of 20 mM of bis-trispropane and 0.4 mM of Tris-HCl buffered at pH 8.0 containing 1.2 mM of MgSO₄, 0.1 mM of EDTA, 25 mM of NaCl and 0.03% of CHAPS (3-[(cholamidopropyl)dimethylammonio]-1-propanesulphonate) are incubated with decreasing concentrations of the compound to be tested (from 100 μM to 0.01 nM) and 150 μM of [¹²⁵I]-IL-8. The non-specific binding is determined in the presence of 300 nM of non-labelled IL-8. After 60 minutes of incubation at ambient temperature, the reaction is stopped by rapid filtration under vacuum on a Whatman GF/C filter which is incubated beforehand for 1 hour at +4° C. in a solution of 1% polyethylenimine (weight/volume) and BSA (<<bovine serum albumin >>) 0.5% (weight/volume). The filters are washed with a solution containing 25 mM of NaCl, 1 mM of MgSO₄, 0.5 mM of EDTA and 10 mM of Tris-HCl buffered at pH 7.4. The radioactivity retained on the filters is measured in a gamma counter.

The affinities of the compounds described in the present invention were also determined by a whole cell binding test. The transfected THP-1 or K-562 cells are placed in suspension in PBS (<<phosphate buffered saline >>) binding test buffer without calcium or magnesium containing 0.5% of BSA (weight/volume), pH 7.4 at the rate of 2.5×10⁶ cells/ml. The competition experiments are carried out in plates of 96 wells of 1 ml in a final volume of 0.25 ml. 0.5×10⁶ cells are incubated with decreasing concentrations of the compound to be tested (100 μM to 0.01 nM) and 150 μM of [¹²⁵I]-IL-8. The non-specific binding is determined in the presence of 300 nM of non-radio-labelled chemokine. After 90 minutes of incubation at +4° C., the reaction is stopped by rapid filtration under vacuum on a Whatman GF/C filter incubated beforehand for 1 hour in a solution of 3% polyethylenimine (weight/volume). The filters are washed with a solution of PBS at pH 7.4 containing 0.5 M of NaCl. The radioactivity contained in the filters is measured in a gamma counter.

The compounds of formula (I) described in the present invention which are tested at the concentration of 10 μM inhibit the binding of [¹²⁵I]-IL-8 onto the CXCR2 receptor by at least 95%.

B) Measurement of the Calcium Flow Rates

The effects of the compounds of the present invention were evaluated on the flow rates of calcium which are induced by IL-8 or Gro-alpha.

THP-1 cells expressing recombinant CXCR2 receptors, U937 cells differentiated with 1% (volume/volume) DMSO (dimethylsulphoxide) or Eol3 cells, are incubated in the presence of a fluorescent indicator, Fura-2 AM, at a concentration of 5 μM for 1 hour at 37° C. After this loading period, the cells are washed and placed in suspension at a concentration of 1×10⁶ cells/ml in a saline solution containing: 136 mM of NaCl, 4.7 nM of KCl, 1.2 mM of MgSO₄, 1.6 mM of CaCl₂, 1.2 mM of KH₂PO₄, 11 mM of glucose, 5 mM of HEPES (N-[2-hydroxyethyl]piperazine-N′-[2-ethanesulphonic acid]), pH 7.4. The cell suspension (2 ml) is placed in a quartz cell and the intensity of fluorescence at 510 nm is measured on an LS50B (Perkin-Elmer) type spectrofluorimeter after alternative excitations at 340 nm and 380 nm. The ratio of the intensities of fluorescence after excitation at 340 nm and 380 nm is determined and the intracellular calcium concentration [Ca²⁺]i is calculated according to the formula:

$$[ {Ca}^{2 +} ] i = {K}_{d} \frac{( R - R min )}{( R max - R )} ( Sf2 / Sb2 )$$

in which
K_{d} represents the affinity constant of the Fura-2 and calcium complex, Rmax is the maximum fluorescence intensity determined after addition of 1 μM of the ionophore Bromo-A23187, Rmin is the minimum ratio determined after addition of 10 mM of EGTA (ethylenebis(oxyethylenenitrilo)-tetraacetic acid) following the addition of ionophore and Sf2/Sb2 is the ratio of the values of fluorescence under excitation at 380 nm determined at Rmin and Rmax respectively.

After a period of stabilisation of 1 minute, for which the base intracellular calcium concentration is determined, the compound to be tested or the control vehicle is added to the cells. After an incubation period of 2 minutes during which the calcium concentration is measured, the cells are stimulated with the various agonists (IL-8 or Gro-alpha). The calcium concentration is measured over 2 minutes.

The compounds of formula (I) described in the present invention inhibit the release of calcium induced by the IL-8 or Gro-alpha.

The activity of the compounds according to the invention, demonstrated during biological tests, is indicative of an antagonist action of the IL-8 and enables envisaging their use in therapeutics.

Compounds (I), as well as their pharmaceutically acceptable salts, solvates and hydrates, for their use as a medicament, are thus another subject of the invention.

Also, according to another of its aspects, the invention relates to the use of the compounds of formula (I), or of one of their pharmaceutically acceptable salts, solvates or hydrates for the preparation of a medicament intended for the preventative or curative treatment in mammals, notably in man, of illnesses which are dependent upon an activation of IL-8 CXCR2 receptor and of the chemokines of the same family, and which are generally characterised by a massive invasion of neutrophils.

Amongst the illnesses which can be treated, in administering a therapeutically sufficient amount of at least one of the compounds of formula (I), the following may be cited: atopic dermatites, osteoarthritis, rheumatoid arthritis, asthma, chronic obstruction of the lungs, acute respiratory distress syndrome, inflammation of the colon, Crohn's disease, ulcerative colitis, apoplectic stroke, myocardial infarction, septic shock, multiple sclerosis, endotoxic shock, psoriasis, septicaemia with gram-negative bacteria, toxic shock syndrome, cardiac, pulmonary or renal ischaemia and reperfusion phenomena, glomerulonephrites, thrombosis, graft versus host reaction, Alzheimer's disease, rejection of allografts, paludism, restenosis, angiogenesis, atherosclerosis, osteoporosis, gingivites, non-physiological release of stem cells from bone marrow, illnesses caused by respiratory viruses, herpes viruses and hepatic viruses, meningitis, encephalic herpes, vasculites of the CNS, cerebral traumatisms, CNS tumours, subarachnoid haemorrhages, post-surgery traurnatisms, mucoviscidosis, prenatal labour, cough, pruritus, interstitial pneumonia, hypersensitiveness, arthritis induced by crystals, arthritis of Lyme's disease, progressive ossifying fibrodysplasia, acute and chronic pancreatites, acute alcoholic hepatites, necrotising enterocolites, chronic sinusites, uveites, polymyosites, vascularites, acne, gastric and duodenal ulcers, coeliac illness, oesophagites, glossites, pulmonary obstructions, pulmonary hyperreactivities, bronchiolites leading to pneumoniae, bronchiectases, bronchiolites, proliferating bronchiolites, chronic bronchites, dyspneae, emphysema, hypercapnia, hypoxemia, hypoxia, surgical reduction of the pulmonary volume, pulmonary fibrosis, pulmonary hypertension, right ventricular hypertrophy, sarcoidosis, attacks of the small bronchioles, errors in ventilation-perfusion, respiratory wheezing, lupi, illnesses associated with a pathological angiogenesis, such as cancer, proliferation of tumour cells and the formation of metastasis in the case, for example, of melanoma and cerebral ischaemia.

The invention thus relates to the use of a compound of formula (I), or of one of its pharmaceutically acceptable salts, solvates or hydrates, for the preparation of a medicament intended for the preventative or curative treatment of atopic dermatites, osteoarthritis, rheumatoid arthritis, asthma, chronic obstruction of the lungs, acute respiratory distress syndrome, inflammation of the colon, Crohn's disease, ulcerative colitis, apoplectic stroke, myocardial infarction, septic shock, multiple sclerosis, endotoxic shock, psoriasis, septicaemia with gram-negative bacteria, toxic shock syndrome, cardiac, pulmonary or renal ischaemia and reperfusion phenomena, glomerulonephrites, thrombosis, graft versus host reaction, Alzheimer's disease, rejection of allografts, paludism, restenosis, angiogenesis, atherosclerosis, osteoporosis, gingivites, non-physiological release of stem cells from bone marrow, illnesses caused by respiratory viruses, herpes viruses and hepatic viruses.

The compounds of formula (I) must be administered in an amount which is sufficient to antagonise the IL-8 in competitively fixing itself onto its receptors. The dose of active principle depends upon the mode of administration and upon the type of pathology and is generally between 0.01 and 10 mg/kg. The compounds of formula (I) can also be combined with another active principle.

Within the context of their therapeutic use, the compounds of formula (I) will in general be administered in various forms, in combination with commonly used excipients. Pharmaceutical compositions containing a compound of formula (I) or one of its pharmaceutically acceptable salts, solvates or hydrates with a pharmaceutically acceptable vehicle, support or excipient, are also another subject of the present invention.

It will be possible for the formulation used to be an oral form, such as capsules or tablets for example which contain the solid active principle in a powdered or micronized form, a syrup or a solution containing the active principle in solution, in suspension, in an emulsion or a in micro-emulsion.

The formulation can also be presented in a form which can be administered for a topical use, e.g. a cream or a lotion or a transdermal device such as an adhesive patch. The active principle can also be formulated for a mode of administration by subcutaneous, intramuscular or intravenous injection.

The following PREPARATIONS and EXAMPLES illustrate the invention without however limiting it. The following abbreviations are used: s=singlet, m=multiplet, d=doublet, t=triplet, quad=quadruplet, q=quintuplet.

Preparation 1

### Methyl Ester of 4-fluoro-ε-oxobenzenehexanoic Acid, Compound IV.1

A suspension of 2.59 g of aluminium chloride in 4 ml of dichloromethane is prepared, which is cooled to −5° C. and a mixture of 0.97 ml of fluorobenzene and 1.31 ml of the methyl ester of 6-chloro-6-oxo-hexanoic acid in 3 ml of dichloromethane is added progressively in keeping the temperature between −4 and −7° C. The temperature is then allowed to rise to 20° C. and, after 15 hours, hydrolysis is effected on acidified, iced water. The mixture is extracted with dichloromethane and the organic phase obtained is washed with water, dried over magnesium sulphate and concentrated under reduced pressure. 2 g of crude product are thus recovered which is purified by chromatography on silica gel in eluting with a petroleum ether/ethyl acetate mixture, 96/4, v/v. 1.26 g of the product sought after are thus obtained in the form of a white powder. (Yield=63%)

M. Pt.=58-59° C.

The following compounds are prepared according to the same method
Methyl ester of 3,4-dichloro-ε-oxobenzenehexanoic acid, compound IV.2; M. Pt.=41-44° C.
Methyl ester of 3,4-difluoro-ε-oxobenzenehexanoic acid, compound IV.3; M. Pt.=41-43° C.
Methyl ester of 4-chloro-3-ethyl-ε-oxobenzenehexanoic acid, compound IV.4

Preparation 2

### Ethyl Ester of 3,4-dichloro-δoxobenzenepentanoic Acid, Compound IV.5

A suspension 5 g of 3,4-dichloro-δ-oxo-benzenepentanoic acid is prepared in 60 ml of ethanol and 1 ml of pure sulphuric acid is added. The mixture is held under reflux under agitation for 5 hours. The reaction mixture is then concentrated under reduced pressure and then taken up in diethyl ether. This organic phase is washed with water, and then with a dilute sodium hydroxide solution, and then again with water. After drying over magnesium sulphate, the solvent is evaporated off under reduced pressure and 2.6 g of the product sought after are obtained in the form of a brown oil (Yield=47%)

¹H NMR (300 MHz, CDCl₃): 8.05 (d, J=1.5 Hz, 1H); 7.80 (dd, J=1.5 Hz, J=8.1 Hz, 1H); 7.56 (d, J=8.1 Hz, 1H); 4.13 (q, J=7.4 Hz, 2H); 3.02 (t, J=6.6 Hz, 2H); 2.42 (t, J=6.6 Hz, 2H); 2.05 (q, J=6.6 Hz, 2H); 1.25 (t, J=7.4 Hz, 3H).

Preparation 3

### Methyl Ester of 4-chloro-3-methoxy-ε-oxobenzenehexanoic Acid, Compound IV.6

### a) Ethyl Ester of α-acetyl-4-chloro-3-methoxy-β-oxobenzenepropanoic Acid

500 μl of ethanol and 48 μl of carbon tetrachloride are added to 237 mg of magnesium and then 4 ml of toluene, 1 ml of ethanol and 920 μl of ethyl acetoacetate are added. The reaction mixture is agitated until the magnesium disappears. The reaction mixture is cooled to −5° C. and then 2 g of (4-chloro-3-methoxy)benzoyl chloride in 1 ml of toluene are added. After 2 hours 30 minutes of agitation at this temperature, the reaction mixture is heated for 30 minutes at 50° C. An ice/sulphonic acid mixture is added. After extraction with toluene, the solvents are evaporated off under reduced pressure.

### b) Ethyl Ester of 4-chloro-3-methoxy-β-oxobenzenepropanoic Acid

A solution of 295 mg of sodium hydroxide in 10 ml of water, 787 mg of ammonium chloride and 1 ml of ammonium hydroxide are added successively to 2.2 g of the compound obtained in a). The reaction mixture is heated at 50° C. for 3 hours, under reflux for 1 hour 30 minutes and at ambient temperature for 48 hours. After extraction with ethyl acetate, the solvents are evaporated off under reduced pressure. A few drops of ethyl acetate are added in order to re-dissolve the precipitate and then petroleum ether is added. The precipitate is filtered off and the filtrate is evaporated under reduced pressure.

### c) Diethyl Ester of 2-(4-chloro-3-methoxybenzoyl)hexanedioic Acid

The residue obtained in b) is added dropwise to a sodium ethoxide solution obtained from 206 mg of sodium in 4 ml of ethanol. The reaction mixture is heated under reflux for 45 minutes and is then cooled to 40° C. and 1.28 ml of ethyl 4-bromobutyrate are added at this temperature. The reaction mixture is heated under reflux for 4 hours 30 minutes. After returning to ambient temperature, the reaction mixture filtered and washed with ethanol. After extraction with ethyl acetate, the solvents are evaporated off under reduced pressure. The residue obtained is purified by chromatography on silica gel in eluting with a petroleum ether/ethyl acetate mixture, 95/5 and then 9/1, v/v.

¹H NMR (300 MHz, CDCl₃): 7.48 (d, 1H); 7.43 (dd, 1H); 7.36 (d, 1H); 4.17 (t, 1H); 4.05 (quad, 2H); 4.01 (quad, 2H); 3.87 (s, 3H); 2.26 (t, 2H); 1.94 (m, 2H); 1.58 (m, 2H); 1.14 (t, 3H); 1.08 (t, 3H).

### d) 4-chloro-3-hydroxy-ε-oxobenzenehexanoic Acid

2.3 ml of hydrobromic acid are added to 390 mg of the compound obtained in c). The reaction mixture is heated under reflux for 4 hours. After returning to ambient temperature and extraction with ethyl acetate, the organic phase is washed with a 10% sodium carbonate aqueous solution. The aqueous phase is acidified to pH=1 with a 1N hydrochloric acid aqueous solution and is then extracted with ethyl acetate. The organic phase is dried and then concentrated under reduced pressure.

¹H NMR (300 MHz, DMSO): 7.50 (m, 3H); 2.58 (m, 2H); 2.25 (m, 2H) 1.55 (m, 4H).

e) Compound IV.6

A mixture of 150 mg of the compound obtained in d), 182 mg of potassium carbonate, and 110 μl of dimethyl sulphate in 2 ml of acetone, is heated under reflux for 15 hours. After filtration, the solvents are evaporated off. After extraction with diethyl ether, the solvents are evaporated off again. The residue is re-dissolved in ethyl acetate and a few drops of petroleum ether are added. The precipitate is filtered off and the filtrate is evaporated.

¹H NMR (300 MHz, CDCl₃): 7.54 (d, 1H); 7.46 (m, 2H); 3.97 (s, 3H); 3.67 (s, 3H); 2.98 (t, 2H); 2.39 (t, 2H); 1.75 (m, 4H).

Preparation 4

### Methyl Ester of 5-bromo-2-(3,4-dichlorophenyl)-1H-indole-3-butanoic Acid, Compound III.1

A mixture of 1.5 g of compound IV.2., 1.74 g of 4-bromophenylhydrazine hydrochloride, and 0.71 g of zinc chloride, is prepared in 10 ml of acetic acid. This mixture is brought up to 65-70° C. and kept under agitation at this temperature for 5 hours. After cooling, 15 ml of water and 20 ml of ethyl acetate are added and the reaction mixture is then filtered. The filtrate is extracted with twice 20 ml of ethyl acetate. The organic phase obtained is washed with water, dried over magnesium sulphate and the solvents are evaporated off under reduced pressure. 1.55 g of a beige solid are obtained.

M. Pt.=112-114° C.

The following compounds are prepared according to the same method
Methyl ester of 2-(3,4-difluorophenyl)-5-iodo-1H-indole-3-butanoic acid, compound III.2; M. Pt.=118-120° C.
Methyl ester of 2-(4-chlorophenyl)-5-iodo-1H-indole-3-butanoic acid, compound III.3; M. Pt.=160-165° C.
Methyl ester of 5-bromo-2-(4-fluorophenyl)-1H-indole-3-butanoic acid, compound III.4; M. Pt.=96-98° C.
Ethyl ester of 2-(3,4-dichlorophenyl)-5-iodo-1H-indole-3-propanoic acid, compound III.5; M. Pt.=135-138° C.
Methyl ester of 2-(5-chloro-2-thienyl)-5-iodo-1H-indole-3-butanoic acid, compound III.6

¹H NMR (300 MHz, DMSO) 11.50 (s, 1H); 7.92 (d, 1H); 7.37 (dd, 1H); 7.33 (d, 1H); 7.24 (d, 1H); 7.18 (d, 1H); 3.58 (s, 3H); 2.85 (t, 2H) 2.39 (t, 2H); 1.82 (q, 2H).
Methyl ester of 2-(4-chloro-3-ethylphenyl)-5-iodo-1H-indole-3-butanoic acid, compound III.7

¹H NMR (300 MHz, CDCl₃): 7.97 (s, 1H); 7.87 (d, 1H); 7.39 (dd, 1H); 7.36. (d, 1H); 7.33 (d, 1H); 7.23 (dd, 1H); 7.08 (d, 1H); 3.55 (s, 3H); 2.75 (m, 4H); 2.27 (t, 2H); 1.93 (q, 2H); 1.22 (t, 3H).
Methyl ester of 2-(4-chloro-3-methoxyphenyl)-5-iodo-1H-indole-3-butanoic acid, compound III.8

¹H NMR (300 MHz, DMSO): 11.4 (s, 1H); 7.95 (s, 1H); 7.55 (d, 1H); 7.38 (dd, 1H); 7.32 (d, 1H); 7.24 (d, 1H); 7.21 (dd, 1H); 3.95 (s, 3H); 3.58 (s, 3H); 2.85 (t, 2H); 2.40 (t, 2H); 1.85 (m, 2H).

Preparation 5

### Methyl Ester of 2-(3,4-dichlorophenyl)-5-cyano-1H-indole-3-butanoic acid, compound II.1

A mixture of 1.6 g of the compound III.1, 2.66 g of cuprous cyanide and 7 ml of N-methyl-2-pyrrolidone is prepared which is heated under reflux for 16 hours. The reaction mixture is then cooled and 30 ml of water are added. The mixture is kept under agitation at ambient temperature for 15 minutes and 20 ml of ethylenediamine are then added. The mixture is then extracted with twice 40 ml of toluene and the combined organic phases are washed with thrice 30 ml of a saturated sodium chloride solution and are then dried over magnesium sulphate. After filtration, the solvents are evaporated off under reduced pressure. The residue of evaporation is dissolved in ethyl acetate and a few drops of cyclohexane are then added. The product sought after is filtered off and is dried under reduced pressure; M. Pt.=158-160° C.

The following compounds are prepared according to the same method
Methyl ester of 5-cyano-2-(3,4-difluorophenyl)-1H-indole-3-butanoic acid, compound II.2;

¹H NMR (300 MHz, CDCl₃): 8.3 (s, 1H); 7.99 (s, 1H); 7.45 (dd, 1H) 7.42 (d, 1H); 7.36 (m, 1H); 7.28 (m, 2H); 3.65 (s, 3H); 2.88 (t, 2H); 2.37 (t, 2H); 2.00 (q, 2H).
Methyl ester of 2-(4-chlorophenyl)-5-cyano-1H-indole-3-butanoic acid, compound II.3; M. Pt.=135-137° C.
Methyl ester of 5-cyano-2-(4-fluorophenyl)-1H-indole-3-butanoic acid, compound II.4;

¹H NMR (300 MHz, CDCl₃): 7.98 (s, 1H); 7.53 (m, 2H); 7.42 (m, 2H); 7.19 (m, 2H); 3.60 (s, 3H); 2.88 (t, 2H); 2.36 (t, 2H); 2.00 (q, 2H).
Ethyl ester of 5-cyano-2-(3,4-dichlorophenyl)-1H-indole-3-propanoic acid, compound II.5; M. Pt.=148-151° C.
Methyl ester of 2-(5-chloro-2-thienyl)-5-cyano-1H-indole-3-butanoic acid, compound II.6; M.Pt. 143-144° C.
Methyl ester of 2-(4-chloro-3-ethylphenyl)-5-cyano-1H-indole-3-butanoic acid, compound II.7

¹H NMR (300 MHz, DMSO): 11.90 (s, 1H); 8.17 (s, 1H); 7.61 (d, 1H); 7.57 (d, 1H); 7.50 (m, 3H); 3.55 (s, 3H); 2.88 (t, 2H); 2.79 (quad, 2H); 2.40 (t, 2H); 1.86 (q, 2H); 1.25 (t, 3H).

Preparation 6

### Methyl Ester of 5-iodo-1H-indole-3-butanoic Acid, Compound VII.1

Compound VII.1 is prepared from 4-iodophenylhydrazine and the methyl ester of 6-oxohexanoic acid according to a method which is analogous to that described in PREPARATION 4.

¹H NMR (300 MHz, DMSO) 10.95 (s, 1H); 7.85 (s, 1H); 7.32 (dd, 1H); 7.19 (d, 1H); 7.13 (d, 1H); 3.59 (s, 3H); 2.66 (t, 2H); 2.35 (t, 2H) 1.85 (q, 2H).

Preparation 7

### Methyl Ester of 5-cyano-1H-indole-3-butanoic Acid, Compound VI.1

A mixture of 1.586 g of compound VII.1, 602 mg of potassium cyanide, 88 mg of copper iodide and 267 mg of tetrakis(triphenylphosphine)palladium in 6 ml of tetrahydrofuran is heated under reflux under agitation for 8 hours. 88 mg of copper iodide and 267 mg of tetrakis(triphenylphosphine)palladium are added again, after 2 hours and 6 hours of agitation. After returning to ambient temperature, 200 ml of ethyl acetate are added and the mixture is filtered on celite. The organic phase is washed twice with water and with a saturated sodium chloride solution and then dried over magnesium sulphate. The solvents are evaporated off under reduced pressure. The residue obtained is purified by chromatography on silica gel in eluting with a petroleum ether/ethyl acetate mixture, 7/3, v/v; M. Pt. 75-76° C.

Preparation 8

### Methyl Ester of 2-bromo-5-cyano-1H-indole-3-butanoic Acid, Compound V.1.

A solution 3 g of compound VI.1 is prepared in 125 ml of carbon tetrachloride and 2.56 g N-bromosuccinimide are added. The reaction mixture is held under reflux under agitation for 4 hours and then cooled to ambient temperature. 200 ml of ethyl acetate and 200 ml of hot water are added. The organic phase is washed with hot water, and then dried over magnesium sulphate. The solvents are evaporated off under reduced pressure. The residue obtained is taken up in petroleum ether and dichloromethane. After removal of the petroleum ether, the precipitate obtained is filtered and washed with toluene. The filtrate is evaporated and the residue obtained is purified by chromatography on silica gel in eluting with the aid of a petroleum ether/ethyl acetate mixture, 8/2, v/v; M. Pt.=105-106° C.

Preparation 9

### Methyl Ester of 2-(4-chloro-3-methylphenyl)-5-cyano-1H-indole-3-butanoic Acid, Compound II.8

A solution of 73 mg of compound V.1 and 58 mg of 4-chloro-3-methylphenylboronic acid is prepared in 4.7 ml of methanol and 4.7 ml of toluene. 29 mg of lithium chloride, 13 mg of tetrakis(triphenylphosphine)-palladium and 0.57 ml of a 1M sodium carbonate solution are then added under agitation. The reaction mixture is then held under reflux under agitation for 1 hour and the solvents are then evaporated off under reduced pressure. The residual solid is purified by chromatography on silica gel in eluting with a petroleum ether/ethyl acetate mixture, 85/15 v/v.

¹H NMR (300 MHz, DMSO): 11.80 (s, 1H); 8.17 (s, 1H); 7.65 (s, 1H); 7.58 (d, 1H); 7.48 (m, 3H); 3.55 (s, 3H); 2.88 (t, 2H); 2.38 (t, 2H); 1.89 (q, 2H).

The following compounds are prepared according to an analogous method:
Methyl ester of 5-cyano-2-(4-fluoro-3-methylphenyl)-1H-indole-3-butanoic acid, compound II.9;

¹H NMR (300 MHz, DMSO): 11.88 (s, 1H); 8.13 (s, 1H); 7.58 (d, 1H); 7.54 (m, 3H); 7.31 (m, 1H); 3.58 (s, 3H); 2.88 (t, 2H); 2.38 (t, 2H); 1.86 (q, 2H).
Methyl ester of 2-[4-chloro-3-(trifluoromethyl)phenyl]-5-cyano-1H-indole-3-butanoic acid, compound II.10;
Methyl ester of 2-(3-chloro-4-fluorophenyl)-5-cyano-1H-indole-3-butanoic acid, compound II.11;

¹H NMR (300 MHz, DMSO): 11.90 (s, 1H); 8.20 (s, 1H); 7.85 (d, 1H); 7.66 (m, 1H); 7.61 (d, 1H); 7.49 (m, 2H); 3.55 (s, 3H); 2.88 (t, 2H); 2.40 (t, 2H); 1.85 (q, 2H).
Methyl ester of 2-(4-chloro-3-fluorophenyl)-5-cyano-1H-indole-3-butanoic acid, compound II.12;

¹H NMR (300 MHz, DMSO): 11.93 (s, 1H); 8.21 (s, 1H); 7.76 (d, 1H); 7.68 (dd, 1H); 7.50 (m, 3H); 3.55 (s, 3H); 2.90 (t, 2H); 2.39 (t, 2H); 1.84 (q, 2H).
Methyl ester of 2-(4-nitrophenyl)-5-cyano-1H-indole-3-butanoic acid, compound II.13;

¹H NMR (300 MHz, CDCl₃): 12.10 (s, 1H); 8.38 (d, 2H); 8.27 (s, 1H) 7.95 (d, 2H); 7.54 (m, 2H); 3.56 (s, 3H); 2.95 (t, 2H); 2.42 (t, 2H); 1.88 (q, 2H).
Methyl ester of 2-(4-cyanophenyl)-5-cyano-1H-indole-3-butanoic acid, compound II.14; M. Pt.=149-151° C.
Methyl ester of 2-[4-(trifluoromethoxy)phenyl]-5-cyano-1H-indole-3-butanoic acid, compound II.15; M. Pt.=142-143° C.

Preparation 10

### Methyl Ester of 2-(4-chloro-3-methoxyphenyl)-5-cyano-1H-indole-3-butanoic Acid, Compound II.16

Compound II.16 is prepared from compound III.8 according to a method which is analogous to that of PREPARATION 7.

¹H NMR (300 MHz, DMSO): 11.90 (s, 1H); 8.19 (s, 1H); 7.58 (d, 1H); 7.52 (d, 1H); 7.48 (dd, 1H); 7.35 (dd, 1H); 7.24 (dd, 1H); 3.98 (s, 1H); 3.57 (s, 3H); 2.91 (t, 2H); 2.40 (t, 2H); 1.88 (m, 2H).

### EXAMPLE 1

### 2-(3,4-dichlorophenyl)-5-cyano-1H-indole-3-butanoic Acid

A mixture of 80 mg of compound II.1 is prepared in 3 ml of dioxane. 1 ml of a 1N sodium hydroxide solution in water is added and the reaction mixture is held under reflux for 1 hour 30 minutes. The solvent is then removed under reduced pressure and the residue is taken up in 3 ml of water. The solution obtained is acidified with 1N hydrochloric acid to pH=1. The precipitate is separated off by filtration and is purified on silica gel in eluting with a dichloromethane/methanol mixture, 9/1, v/v; M. Pt.=190-195° C.

According to an analogous method, EXAMPLES 2 to 16, presented in TABLE 1 below, are prepared:

TABLE 1

| | |
|---|---|
| 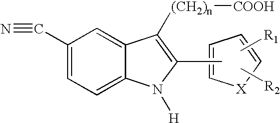 | (I) |

| EXAM- | | | M.Pt.; |
|---|---|---|---|
| PLE | 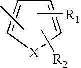 | n | ° C. |
| 2 | 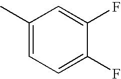 | 3 | 223-224 |
| 3 | 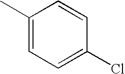 | 3 | 225-230 |
| 4 | 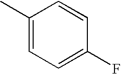 | 3 | 167-169 |
| 5 | 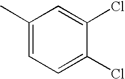 | 2 | 198-200 |
| 6 | 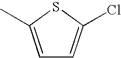 | 3 | 204-206 |
| 7 | 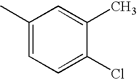 | 3 | 196-198 |
| 8 | 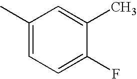 | 3 | 179-180 |
| 9 | 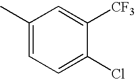 | 3 | 100-102 |
| 10 | 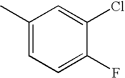 | 3 | 195-197 |
| 11 | 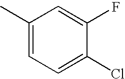 | 3 | 255-257 |
| 12 | 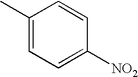 | 3 | 296 |
| 13 | 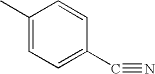 | 3 | 262-264 |
| 14 | 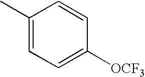 | 3 | 198-199 |
| 15 | 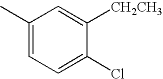 | 3 | 143-146 |
| 16 | 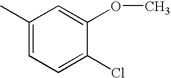 | 3 | 187-189 |

1. Compounds of formula (I): 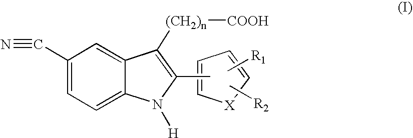
wherein:
X is a double bond —C═C— or a sulphur atom,
R₁ and R₂ represent, each one independently of the other, hydrogen, halogen, (C₁-C₃)alkyl, (C₁-C₃)alkoxy, trifluoromethyl, trifluoromethoxy, cyano or nitro,
n is 2 or 3,
as well as their pharmaceutically acceptable salts, solvates and hydrates.

2. Compounds according to claim 1 having the formula (Ia) 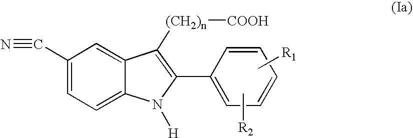
wherein:
R₁ and R₂ represent, each one independently of the other, hydrogen, halogen, (C₁-C₃)alkyl, (C₁-C₃)alkoxy, trifluoromethyl, trifluoromethoxy, cyano or nitro,
n is 2 or 3,
as well as their pharmaceutically acceptable salts, solvates and hydrates.

3. Compounds according to claim 2 having the formula 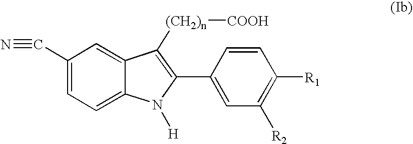
wherein:
R₁ and R₂ represent each one independently of the other, hydrogen, halogen, (C₁-C₃)alkyl, (C₁-C₃)alkoxy, trifluoromethyl, trifluoromethoxy, cyano or nitro,
n is 2 or 3,
as well as their pharmaceutically acceptable salts, solvates and hydrates.

4. Compounds according to claim 1, wherein R₁ and R₂ represent, each independently, hydrogen, chlorine, fluorine, (C₁-C₂)alkyl, methoxy, trifluoromethyl, trifluoromethoxy, cyano or nitro.

5. Compounds according to claim 4, wherein R₁ and R₂ represent, each independently, hydrogen, chlorine, fluorine or methyl.

6. Compounds according to claim 1 wherein n is 3.

7. Compounds according to claim 1 wherein R₁ is chlorine or fluorine.

8. Compounds of formula (II): 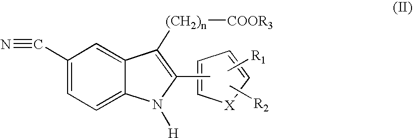
wherein:
R₁ and R₂ represent, each one independently of the other, hydrogen, halogen, (C₁-C₃)alkyl, (C₁-C₃)alkoxy, trifluoromethyl, trifluoromethoxy, cyano or nitro,
n is 2 or 3,
R₃ is (C₁-C₄)alkyl.

9. (canceled)

10. A pharmaceutical composition comprising a compound according to claim 1 with a pharmaceutically acceptable vehicle, support or excipient.

11. A method for treating illnesses which are dependent upon the activation of the interleukin CXCR2 receptor and of the chemokines of the same family, comprising administering a therapeutically effective amount of a compound according to claim 1.

12. A method according to claim 11 wherein said illness is selected from the group consisting of atopic dermatites, osteoarthritis, rheumatoid arthritis, asthma, chronic obstruction of the lungs, acute respiratory distress syndrome, inflammation of the colon, Crohn's disease, ulcerative colitis, apoplectic stroke, myocardial infarction, septic shock, multiple sclerosis, endotoxic shock, psoriasis, septicaemia with gram-negative bacteria, toxic shock syndrome, cardiac, pulmonary or renal ischaemia and reperfusion phenomena, glomerulonephrites, thrombosis, graft versus host reaction, Alzheimer's disease, rejection of allografts, paludism, restenosis, angiogenesis, atherosclerosis, osteoporosis, gingivites, non-physiological release of stem cells from bone marrow, illnesses caused by respiratory viruses, herpes viruses and hepatic viruses.
